(19) United States
(12) Patent Application Publication
(10) Pub. No.: US 2016/0052903 A1
(43) Pub. Date: Feb. 25, 2016

(54) 5-(HYDROXYMETHYL) FURAN-2-CARBALDEHYDE (HMF) SULFONATES AND PROCESS FOR SYNTHESIS THEREOF

(71) Applicant: ARCHER DANIELS MIDLAND COMPANY, Decatur, IL (US)

(72) Inventor: Kenneth STENSRUD, Decatur, IL (US)

(73) Assignee: Archer Daniels Midland Company, Decatur, IL (US)

(21) Appl. No.: 14/780,203

(22) PCT Filed: Apr. 25, 2014
(86) PCT No.: PCT/US14/35395

§ 371 Date: Sep. 25, 2015

Related U.S. Application Data
(60) Provisional application No. 61/816,847, filed on Apr. 29, 2013.

(51) Int. Cl. C07D 307/46; C07D 307/52; C07D 307/54; C07C 51/41

(52) U.S. Cl. CPC C07D 307/46; C07C 51/41; C07D 307/52; C07D 307/54

(57) ABSTRACT

5-(hydroxymethyl)furan-2-carbaldehyde (HMF)-sulfonates and a method of preparing the same are described. The method involves reacting a mixture of 5-(hydroxymethyl)furfural (HMF), with at least one of a) a trifluoromethanesulfonate anhydride (triflate), b) a p-toluene-sulfonyl halide (tosylate), and c) methane-sulfonyl halide (mesylate), and a reagent of either 1) a nucleophilic base or 2) a combination of a non-nucleophilic base and a nucleophile. The HMF-sulfonates (e.g., triflate, tosylate, mesylate, etc. analogs of HMF) can serve as precursor materials from which various derivative compounds can be synthesized.

### BENEFIT OF PRIORITY

The present application claims benefit of priority from U.S. Provisional Application No. 61/816,847, filed Apr. 29, 2013, the contents of which are herein incorporated.

### FIELD OF INVENTION

The present disclosure relates to furanic sulfonate molecules, to particular methods by which such molecules are prepared, to certain derivative compounds or materials made from such molecules, and methods for making the derivative compounds.

### BACKGROUND

Biomass contains carbohydrates or sugars (i.e., hexoses and pentoses) that can be converted into value added products. Production of biomass-derived products for non-food uses is a growing industry. Rio-based fuels are an example of an application with growing interest. Another application of interest is the use of biomass as feedstock for synthesis of various industrial chemicals from renewable hydrocarbon sources.

In recent years, an increasing effort has been devoted to find ways to utilize biomass as feedstock for the production of organic chemicals because of its abundance, renewability, and worldwide distribution. When considering possible downstream chemical processing technologies, the conversion of sugars to value-added chemicals is very important. Recently, the production of furan derivatives from sugars has become exciting in chemistry and in catalysis studies, because it aids major routes for achieving sustainable energy supply and chemicals production.

The compound 5-(hydroxymethyl)furfural (HMF) is an important intermediate substance readily made from renewable resources, specifically carbohydrates.

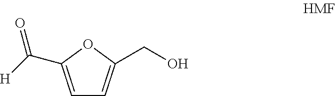

HMF is a suitable starting material for the formation of various furan ring derivatives that are known intermediates for a variety chemical syntheses, and as potential substitutes for benzene based compounds ordinarily derived from petroleum resources. Due to its various functionalities, it has been proposed that HMF could be utilized to produce a wide range of products such as polymers, solvents, surfactants, pharmaceuticals, and plant protection agents. As substitutes, one may compare derivatives of HMF to chemicals with the corresponding benzene-based rings or to other compounds containing a furan or tetrahydrofuran. HMF and 2,5-disubstituted furans and tetrahydrofuran derivatives, therefore, have great potential in the field of intermediate chemicals from renewable agricultural resources. In order to compete with petroleum based derivatives, however, preparation of HMF derivatives from common agricultural source materials, such as sugars, must be economical.

Until recently, furanics have not been commercialized because large-scale production of furanic intermediates has not been cost-effective. The common dehydration route of fructose to HMF generates many side products, making subsequent purification severely cumbersome yet indispensable. Various different processes have been advanced for the catalytic conversion of sugar to furan chemicals. (See generally, X. Tong et al., “Biomass into Chemicals: Conversion of Sugars to Furan Derivatives by Catalytic Processes,” APPLIED CATALYSIS A: GENERAL 385 (2010) 1-13.)

The relevance or appeal of HMF as a renewable, bio-based feedstock is underscored in several recent reviews, such as: Robert-Jan van Putten et al., “Hydroxymethylfurfural, A Versatile Platform Chemical Made from Renewable Resources”, Chemical Reviews 2013 ASAP; K. S. Arias, et al., “From Biomass To Chemicals: Synthesis of Precursors of Biodegradable Surfactants from 5-Hydroxymethylfurfural”, Chem Sus Chem 2013 6 123-131; Dutta, Saikat; De, Sudipta; Saha, Basudeb “A Brief Summary of the Synthesis of Polyester Building-Block Chemicals and Biofuels from 5-Hydroxymethylfurfural” Chem Plus Chem 2012, 77(4), 259-272; or Amarasekara, Ananda S. “5-hydroxymethylfurfural based polymers”, Renewable Polymers (2012), 381-428; Rosatella, Andreia A.; Simeonov, Svilen P.; Frade, Raquel F. M.; Afonso, Carlos A. M. “5-Hydroxymethylfurfural (HMF) as a building block platform: Biological properties, synthesis and synthetic applications”, Green Chemistry (2011), 13(4), 754-793.) Nonetheless, one of the concerns with HMF, is that HMF itself is rather unstable and tends to polymerize and/or oxidize with prolonged storage. Furthermore, HMF is also somewhat difficult to isolate and that it has limited uses as a chemical per se, other than as a source for making derivatives.

Currently, one is not able to easily convert HMF directly into many derivative compounds. Typically, one needs to perform a series of inter-related hydrogenation or hydrogenolysis of HMF to convert formic intermediates into esters or ethers, which in turn, one can transform more readily into useful end products. Nonetheless, the conversion routes from esters or ethers tend to be narrow in scope. In view of the current shortcomings and great potential of HMF as a renewable chemical material, a need exists for an alternative means of working with HMF. The present invention can address this need to provide a modified HMF molecule which is both stable and enables one to perform a variety of chemical reactions directly from the modified HMF molecule. The modified HMF molecule can increase the utility of HMF and support the interest to develop better ways of making building blocks for the emerging market of green materials and renewable energy.

### SUMMARY OF THE INVENTION

The present invention pertains, in part, to a method for preparing 5-(hydroxymethyl)furan-2-carbaldehyde (HMF) sulfonates. The method involves reacting a mixture of a 5-(hydroxymethyl)furfural (HMF) with a sulfonate species, and a reagent of either 1) a nucleophilic base or 2) a combination of a non-nucleophilic base and a nucleophile. In certain embodiments, the sulfonate species can be at least one of: a) a trifluoromethanesulfonate anhydride (triflate), b) a p-toluene-sulfonyl halide (tosylate), c) methane-sulfonyl halide (mesylate), d) ethanesulfonyl halide (esylate), and e) benzenesulfonyl halide (besylate).

In another aspect, the present invention relates to the HMF-sulfonates prepared according to the method described herein, and their use as precursor chemicals for subsequent modification or derivatization into other chemical compounds. In the particular, the HMF-sulfonates include: a) (5-formylfuran-2-yl)methyl methanesulfonate (HMF-mesylate); b) (5-formylfuran-2-yl)methyl trifluoromethanesulfonate (HMF-triflate); c) (5-formylfuran-2-yl)methyl ethanesulfonate (HMF-esylate); d) (5-formylfuran-2-yl)methyl 4-methylbenzenesulfonate (HMF-tosylate); and e) (5-formylfuran-2-yl)methyl benzenesulfonate (HMF-besylate).

In other aspects, the present invention relates to a process for making certain furanic derivative compounds of HMF-sulfonates, and the derivative compounds that are synthesized directly from HMF-sulfonates by means of transforming reactions, such as alkylation, amination, esterification, halogenation, olefination, oxidation, reduction, or thiolation, etc.

### DETAILED DESCRIPTION OF THE INVENTION

### SECTION I

### DESCRIPTION

HMF-sulfonates can open new pathways that enable better use of as a starting material and more convenient chemical synthesis. HMF-sulfonates are useful as precursor chemical materials for a variety of potential compounds, including for instance: polymers, alcohols, organic acids, amphiphiles, surfactants, or solvents. A significant advantage of using sulfonate analogs of HMF over HMF in chemical reactions is that the sulfonate moiety allows for facile introduction of new, useful functionalities to an HMF molecule that otherwise would be very difficult or impossible to achieve via direct derivation of HMF. For example, preparation of furanic derivatives having long chain, unsaturated fatty acids would be an inefficient, low-yielding (e.g., <5%) process if performed directly from HMF. Using current HMF-processing methods, one would not be able to modify and directly halogenate, aminate, thiolate, longer-chain alkylate or olefinate the HMF molecule within two reaction steps, and obtain yields in significant quantities sufficient for potential commercial uses.

HMF-sulfonates can provide an advantage to supervening conversions. HMF-sulfonate reactions are largely quantitative; hence, they can generate with minimal loss, high yields of a target derivative product. Additionally, HMF-sultanates can help control and enhance selectivity of certain reactions. As demonstrated in the accompanying examples, the HMF-sulfonates enable one to selectively react at either the sultanate moiety or the aldehyde moiety. The sulfonate moiety can either preserve the aldehyde functional group on the HMF molecule, or direct the chemical reaction toward the methylene position. For example, if a target compound is a mono-aldehyde prepared by an oxidation reaction, to derivatize directly from HMF would be problematic without the sulfonate moiety, as both the —OH and aldehyde moieties would oxidize without any selectivity.

Further in another example, to perform a Fisher esterification directly with HMF would be unlikely, because one must use an acid catalyst and high temperatures. As mentioned above, HMF is inherently unstable, it will readily polymerize at elevated temperatures, even in the absence of air. Conversion of HMF into HMF-sulfonates, however, can significantly stabilize HMF because the —OH moiety is changed to a sulfonate, obviating the molecule's capacity to polymerize with the aldehyde moiety of another HMF molecule.

### A. Preparation of HMF-Sulfonates

The present disclosure provides, in part, an efficient and facile process for synthesizing 5-(hydroxymethyl)furan-2-carbaldehyde (HMF) sulfonate (i.e., HMF sulfonate) under relatively mild conditions. The process involves reacting a mixture of 5-(hydroxymethyl)furfural (HMF) with at least a sulfonate species, and a reagent of either 1) a nucleophilic base or 2) a combination of a non-nucleophilic base and a nucleophile, as two separate reagents. One can use a variety of sulfonates, such as mesylate (methanesulfonate),

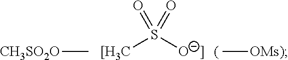

triflate (trifluoromethanesulfonate),

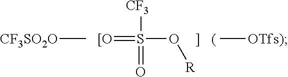

tosylate (p-toluenesulfonate),

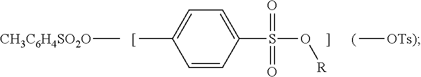

esylate (ethanesulfonate),

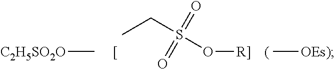

besylate (benzenesulfonate),

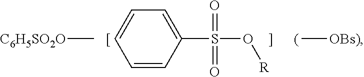

or other sulfonate species without limitation.

The present synthesis process can result in copacetic yields of corresponding HMF sulfonates, as demonstrated in the accompanying examples. The process is able to produce HMF sulfonates in reasonably high molar yields of at least 50% from the HMF starting materials, typically about 55%-70%. With proper control of the reaction conditions and time, one can achieve a yield of about 80%-90% or better of the HMF sulfonates. HMF can be obtained either commercially or synthesized from relatively inexpensive, widely-available biologically-derived feedstocks.

Scheme 1 illustrates an example of a first reaction pathway using a nucleophilic base. According to this first embodiment or pathway, the process involves reacting initially a nucleophilic base with the sulfonate (i.e., trifluoromethanesulfonate anhydride) to generate a reactive intermediate, then adding HMF to the reaction to generate the HMF triflate.

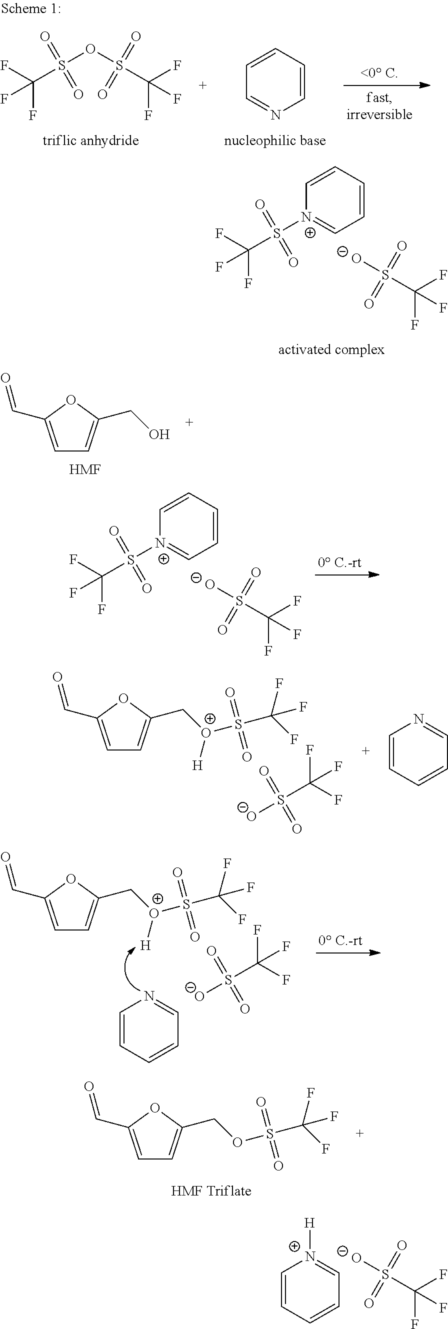

This reaction exhibits relatively fast kinetics and generates an activated triflic complex. This reaction is essentially irreversible, as the liberated triflate is entirely non-nucleophilic. The triflic complex then reacts readily with the HMF, forming an HMF-triflate with concomitant release and protonation of the nucleophilic base. The tosylate, mesylate or other sulfonate species can also be used, but they tend to react more slowly in comparison to the triflate.

The single reactive species is both a nucleophile and a base that can deprotonate the hydroxyl-group of the HMF anhydride. Different reagents can be employed as a nucleophilic base in the present synthesis process. Some common nucleophilic bases that can be used may include, for example: pyridine, derivative thereof, or structurally similar entity, such as dimethyl-aminopyridine (DMAP), imidazole, pyrrolidine, and morpholine. In particular embodiments, pyridine is favored because of its inherent nucleophilic and alkaline attributes, relative low cost, and ease of removal (e.g., evaporation, water solubility, filtration (protonated form) from solution.

In certain protocols, the synthesis process involves reacting the trifluoromethanesulfonic anhydride with the nucleophilic base prior to an addition of the HMF so as to activate the anhydride and form a labile, ammonium (e.g., pyridinium) intermediate (Scheme 2), which it is believed enables the poorly nucleophilic hydroxyl group of the HMF to directly substitute, forming the HMF-triflate compound and to both release and protonate the nucleophilic base.

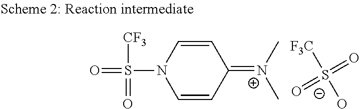

### N-methyl-N-(1-((trifluoromethyl)sulfonyl)pyridin-4(1H)-ylidene)methanaminium trifluoromethanesulfonate

As a second-order reaction, the reaction is conducted at a relatively low initial temperature, which permits one to control the reaction kinetics to produce a single desired compound and helps minimize the generation of a mixture of different byproducts in significant amounts. In other words, the cool to cold initial temperature helps lower the initial energy of the system, which increases control of the kinetics of the reaction, so that one can produce selectively more of the HMF-triflate species than side products.

In the reaction between the triflate and HMF, an interesting phenomenon is observed. During the addition of Tf₂O, a temperature higher than −10° C. will generate dark brown solutions with profuse solid matter (humins) and result in relatively low HMF yields (i.e., <15-20%). This suggests that competitive side reactions will tend to occur at temperatures greater than −10° C. For example, at −10° C. to −15° C., an intensely yellow solution without solids was discerned after all of the triflate was added. Hence, the reaction should be conducted at an initial temperature not greater than about −10° C. or −12° C. In certain embodiments, the initial temperature is typically in a range between about −15° C. or −17° C. and about −78° C. or −80° C. In preferred embodiments, the initial temperature can range between about −20° C. or −25° C. and about −60° C. or −75° C. Particular temperatures can be from about −22° C. or −25° C. to about −65° C. or −70° C. (e.g., −27° C. −30° C., −32° C., −36° C., −38° C., −40° C., −45° C., −50° C., −55° C. or −57° C.). (As used herein, the term “about” used in reference to a value means the referenced value plus or minus the degree of error inherent to an instrument used to measure the value.) Generally for the other sulfonate species—meslyate, tosylate etc.—the synthesis reaction may require less rigorously controlled conditions, and can be conducted at a higher initial temperature of up to about 10° C., typically about 1° C. or less.

In the synthesis of HMF sultanates, one should be conscientious about the stringency of reaction conditions such as temperature, rate of addition of the reagents, and the ratio of sulfonate to HMF. For instance, one should maintain a maximum of 1:1 molar equivalent of Tf₂O per HMF. Addition of volumes in excess of 1 molar equivalent (even by an excess of as little as ˜0.03-0.05) of Tf₂O can induce the reaction solution to decolorize and precipitate solids from solution and result in imperceptible HMF-triflate yields. Further, rapid addition of Tf₂O can result in deeply colored solutions accompanied by precipitate formation and negligible yields of HMF-tiflates even at low temperatures of −78° C. To prevent this from happening, one should add the sulfonate to HMF in a slow and gradual manner. As the reaction between sulfonates and HMF is stoichiometric, the addition of the triflate species to HMF should be about 0.03-0.05 or 0.06 equivalents per minute (e.g., about 20-25-30 or 34 microliters of the triflate per minute in a 10 mL vessel (see, Example 1)). Generally, the total amount of sulfonate (e,g., 666 μL in Example 1) to be consumed in the reaction can be introduced over a period of about 20-30-40 minutes. The other sulfonate species appear not to be as sensitive to these kinds of reaction parameters. Hence, the other sulfonate species can be added more rapidly or in some cases all at once.

As the synthesis reaction uses an excess amount of a nucleophilic base, any acid that may be formed in the reaction (e.g., protonated form of isosorbide) immediately will be deprotonated, hence the pH will be alkaline (i.e., greater than 7).

Scheme 3 presents an example of a second reaction pathway which uses the combination of a non-nucleophilic base and a nucleophile. According to this second embodiment or pathway, either a tosylate (as shown) or other sulfonates (e.g., mesylate, esylate, or besylate) reacts directly with the HMF in a non-nucleophilic base-faciliated sulfonation of HMF.

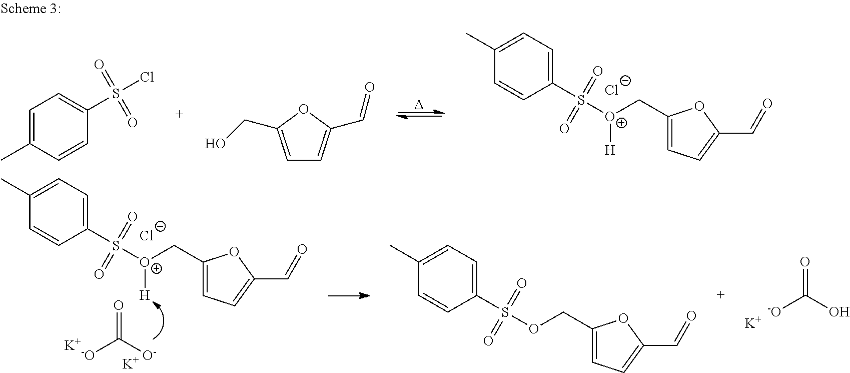

This reaction is reversible and exhibits relatively slow kinetics; hence, heat is added to help promote formation of the intermediate and drive the reaction forward to products. A non-nucleophilic base, such as potassium carbonate, is employed to deprotonate the HMF-toslyate compound. Some common non-nucleophilic bases that may be employed in the reaction include, for example: carbonates, bicarbonates, acetates, or anilines. This reaction is usually performed at about ambient room temperatures (e.g., 20° C.-25° C.) or greater. In some reactions, the temperature can be as high as about 50° C. or 60° C., but typically is about 18° C.-25° C.-30° C. or 40° C. up to about 45° C.-55° C. The specific temperature depends on the type of solvent used in the reaction, and should be controlled to minimize excess side-product formation. As heating is required, the triflate species is not suitable and should not be used with this second pathway.

In the second pathway, a combination of a non-nucleophilic base and a nucleophile is reacted. The non-nucleophilic base can be an amine, including but not limited to triethylamine, N,N-diisopropylethylamine (Hünig's base, (DIPEA or DIEA)), N-methylpyrrolidine, 4-methylmorpholine, and 1,4-diazabicyclo-(2,2,2)-octane (DABCO). In some embodiments, a tertiary amine base is combined with a nucleophilic catalyt, such as strongly nucleophilic 4-dimethylaminopyridine (DMAP). The nucleophile can be present in catalytic amounts, such as 1-5 mole % (0.01 to 0.05 equivalents) or less of the reactive species.

In another embodiment according to the second pathway, when the nucleophile is the alcohol moiety of HMF itself, the reaction involves a non-nucleophilic base and heat, as thermal energy is necessary to transcend a high activation barrier of the transitory, protonated intermediate. This dynamic is illustrated in Scheme 4, which shows HMF reacting with a toslyate chloride to produce HMF-tosylate, and an amine.

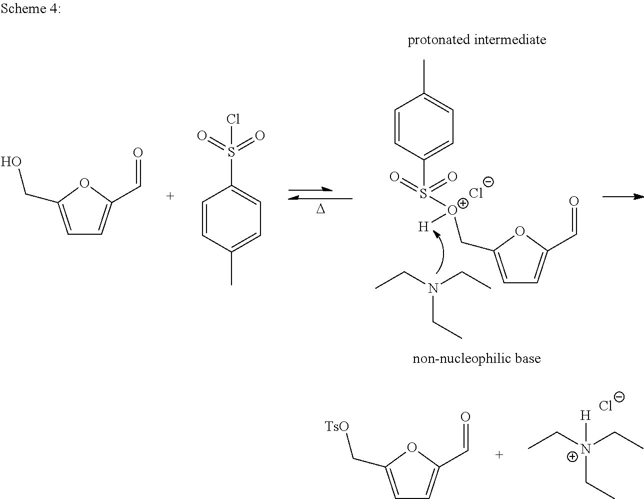

The introduction of heat, however, can increase the probability of generating side products; thus, this second non-nucleophilic base pathway is less favored than the first pathway reacting with a nucleophilic base.

### B.—HMF-Sulfonates

In another aspect, the present invention pertains to an HMF sulfonate prepared according to the present synthesis method. Table 1 lists some of the different HMF-sulfonate compounds.

TABLE 1

| Common Name | IUPAC Name | Structure |
|---|---|---|
| (HMF-mesylate) | (5-formylfuran-2-yl)methyl methanesulfonate | 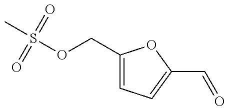 |
| (HMF-triflate) | (5-formylfuran-2-l)methyl trifluoromethanesulfonate | 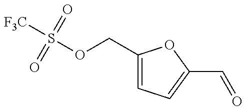 [SMILES: O=Cc1ccc(COS(=O)(=O)C(F)(F)F)o1] |
| (HMF-esylate) | (5-formylfuran-2-yl)methyl ethanesulfonate | 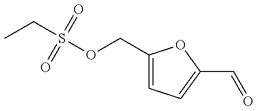 |
| (HMF-tosylate) | (5-formylfuran-2-yl)methyl 4-methylbenzenesulfonate | 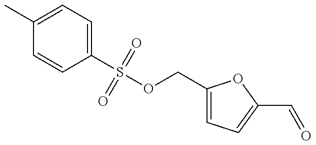 [SMILES: Cc1ccc(S(=O)(=O)OCc2ccc(C=O)o2)cc1] |
| (HMF-besylate) | (5-formylfuran-2-yl)methyl benzenesulfonate | 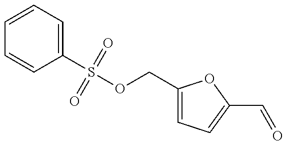 [SMILES: O=Cc1ccc(COS(=O)(=O)c2ccccc2)o1] |

As discussed below, the versatility of HMF sulfonates (e.g., mesolyate, tosylate, triflate, etc. analogs of HMF) makes them useful as a precursor chemical platform from which various different kinds of derivative compounds can be prepared.

### C.—Furanic Derivatives of HMF-Sulfonates

In another aspect, the present disclosure pertains to certain furanic derivative compounds and a method for their preparation. In comparison to HMF, HMF-sulfonates are useful as direct antecedents to a variety of chemical analog compounds. An advantage to the use of HMF-sulfonates is that any desired nucleophilic substitution on HMF is facilely achieved with HMF-sulfonates, which otherwise would be very difficult or impossible with itself. HMF-sulfonates can control the reactivity of the aldehyde moiety depending on the particular reaction. For S_{N}2 chemistry, one can direct the reaction to the sulfonate moiety without involving the aldehyde. In other reactions, such as reductive aminations or olefinations, a reagent will be more reactive towards the aldehyde carbonyl moiety.

Particular HMF-sulfonate species can perform more readily than others in subsequent derivation chemical reactions. For instance, the triflate moiety is one of the best nucleofuges (i.e., leaving groups) in the realm of organic synthesis, permitting both elimination and nucleophilic substitution events to be facilely rendered through light control of reaction conditions, such as temperature, solvent, and stoichiometry. Other sultanate species—melylates, esylates, toslyates, etc.—are less reactive than triflates. Thus, one may select a particular HMF-sulfonate species to tailor its relative degree of reactivity for the desired chemical reaction.

Once HMF sulfonates are synthesized according to the method as described, they can be transformed directly and readily to into other furanic derivative compounds by means of relatively simple reaction processes. For example, one can react the HMF-sulfonate with least one of the following: a) an alkyl- or aryl-magnesium halide (Grignard reagents) or organolithium compound (lithiates), b) an organic acid, c) an alkyl halogen, d) an oxidizing reagent, e) a reducing reagent, f) a Schiff-base, g) a thiol and h) a Wittig reagent to perform, respectively, an alkylation, esterification, halogenation, oxidation, reduction. Schiff-base modification, thiol substitution, or Wittig olefination, such as depicted conceptually in Scheme 5.

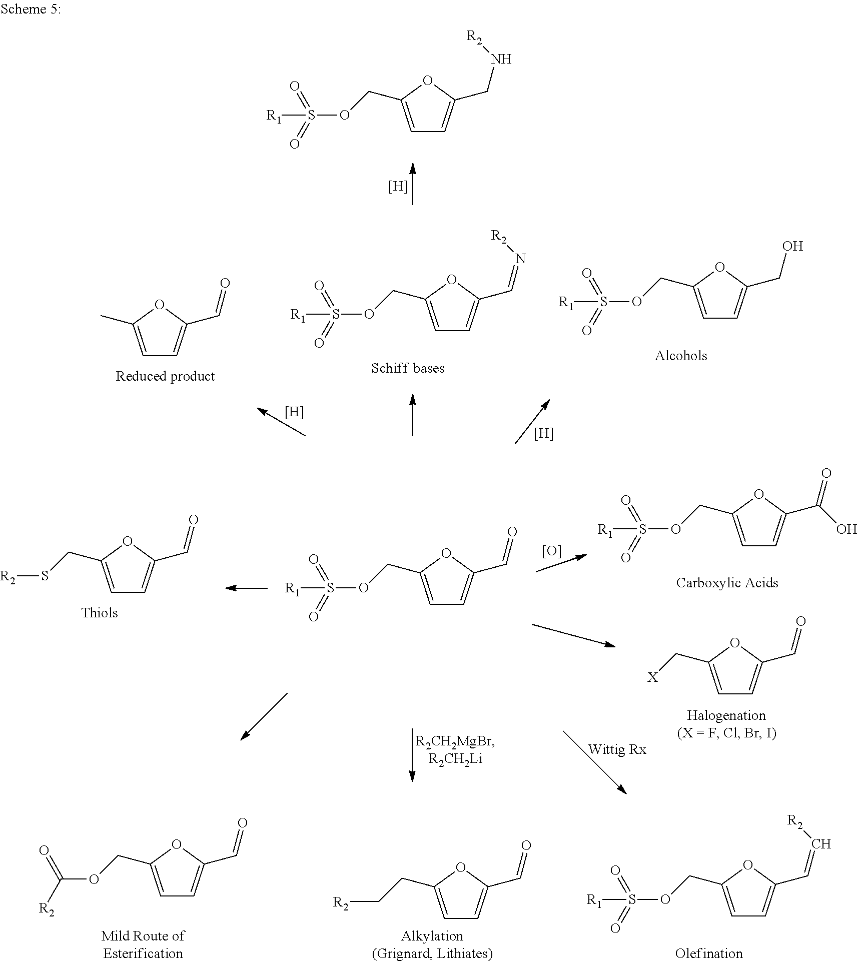

wherein [H] is reduction, [O] is oxidation, R₁ is a —CH₃, —CF₃, —C₆H₄CH₃, —C₂H₅, or —C₆H₅, and R₂ is a alkyl, allyl, or aryl species. Table 2 presents examples of particular furanic derivative compounds that can be made from each type of reaction.

TABLE 2

| Reaction | General Structure | Example IUPAC Name | Example Structure |
|---|---|---|---|
| Alkylation | 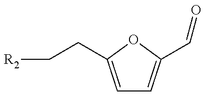 | 5-propylfuran-2-carbaldehyde | 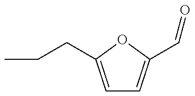 |
| Esterifica- tion | 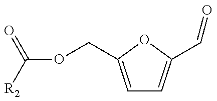 | (5-formylfuran-2-yl)- methylpentanoate | 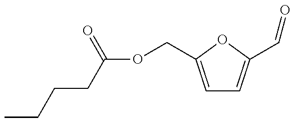 |
| Halogena- tion | 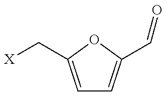 | 5-(iodomethyl)-furan-2- carbaldehyde, | 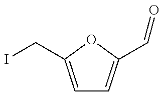 |
| Oxidation | 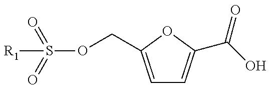 | 5-((((trifluoromethyl)sulfonyl)oxy) methyl)-furan-2-carboxylic acid | 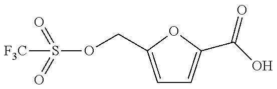 [SMILES: O=C(O)c1ccc(COS(=O)(=O)C(F)(F)F)o1] |
| Aldehyde Reduction | 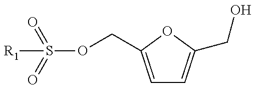 | (5-(hydroxymethyl)furan-2-yl)- methyl benzenesulfonate, | 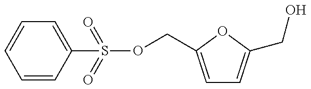 [SMILES: O=S(=O)(OCc1ccc(CO)o1)c1ccccc1] |
| Sulfonate Reduction | | 5-methylfuran-2-carbaldehyde | 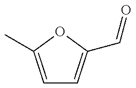 |
| Schiff Base modification | 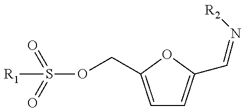 | (Z)-(5-((propylimino)methyl)furan- 2-yl)-methyl methanesulfonate | 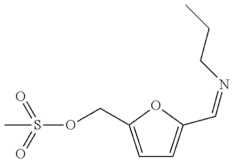 [SMILES: CCC/N=C\c1ccc(COS(C)(=O)=O)o1] |
| Thiol substitution | 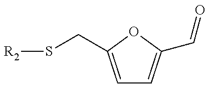 | 5-((pentylthio)methyl)furan-2- carbaldehyde | 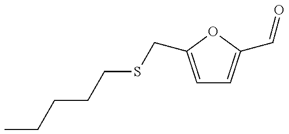 |
| Wittig Olefination | 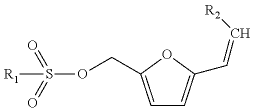 | (Z)-methyl 3-(5- ((tosyloxy)methyl)furan-2-yl) acrylate | 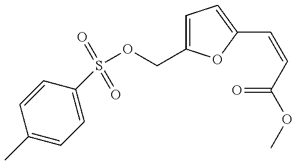 [SMILES: COC(=O)/C=C\c1ccc(COS(=O)(=O)c2ccc(C)cc2)o1] |

Section II, below, presents other examples of furanic derivative compounds that can be synthesized from the present HMF sulfonates. The foregoing list of reactions and the examples are not intended to be an exhaustive catalogue of derivative compounds, but merely a non-limiting illustration of representative derivatives.

### SECTION II

### EXAMPLES

The following examples are provided as illustration of the different aspects of the present disclosure, with the recognition that altering parameters and conditions, for example by change of temperature, time and reagent amounts, and particular starting species and catalysts and amounts thereof, can affect and extend the full practice of the invention beyond the limits of the examples presented.

### A. HMF-Sulfonates

The following examples refer to mesylates, triflates, and tosylates for purposes of illustration; however, the scope of the invention is not necessarily limited to those particular embodiments that incorporate more common or commercially available sulfonate species.

### Example 1

### Synthesis of (5-formylfuran-2-yl)methyl 4-methylbenzenesulfonate, HMF Triflate B

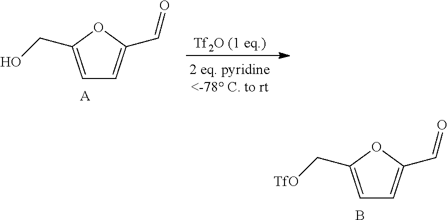

Experimental: An oven dried, 25 mL round bottomed flask equipped with an octagonal PTFE coated magnetic stir bar was charged with 500 mg of HMF A (3.97 mmol), 640 μL of pyridine (7.92 mmol) and 10 mL of anhydrous methylene chloride. The neck of the flask was then capped with a rubber septum fitted with an argon needle inlet and immersed in a saturated dry ice/acetone bath displayed a temperature of −78° C. While stirring and under an argon blanket, 666 μL of triflic anhydride (3.97 mmol) was added drop-wise via syringe over a 30 minute period. After the volume has been added, the reaction mixture was removed from the cooling bath and stirred under ambient temperature for 2 more hours. After this time light yellow solution was observed. The solution was poured into a 250 mL separation funnel, and diluted with 20 mL of methylene chloride and 20 mL of 1N HCl. After vigorous agitation, the lower organic layer was discharged, retained aqueous phase extracted with 15 mL of methylene chloride, organic layers combined, dried with anhydrous magnesium sulfate and concentrated under reduce pressure, affording a 775 mg of orange-hued oil (76% of theoretical). An aliquot of the oil was withdrawn, diluted with methylene chloride, spotted lightly on a pre-cut silica gel plate and developed in a TLC chamber with 100% ethyl acetate as the eluent. A single band was observed, featuring an Rf=0.57. A side-by-side TLC comparison between HMF and product mixture indicated that all the HMF had converted, adduced by the absence of HMF band (Rf=0.48) in the mixture. Another 500 μL aliquot of the product was withdrawn and analyzed by ¹³C and ¹H NMR. ¹H NMR (CDCl₃, 400 MHz) δ (ppm) 9.52 (s 1H), 7.15 (d, J=6.2 Hz, 1H), 6.50 (d, J=6.4 Hz), 1H), 4.56 (s, 2H); ¹³C NMR (CDCl₃, 125 MHz) δ (ppm) 177.91 157.40, 152.94, 122.18, 112.08, 110.18, 64.68. Collectively, the aforementioned data provides cogent proof for the manifestation of HMF triflate B as the primacy product of the reaction.

The target yield is can be actually much greater but some yield may have been lost through a) decomposition when subject to 1N HCl and/or b) partial solubility in the aqueous phase. This loss can be mitigated by means of direct charge of the product mixture onto a pre-fabricated silica gel column, followed by flash chromatography.

### Example 2

### Synthesis of (5-formylfuran-2-yl)methyl 4-methylbenzenesulfonate, HMF Tosylate B

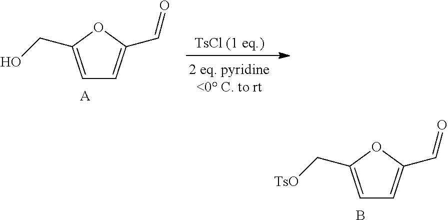

Experimental: An oven dried, 25 mL round bottomed flask equipped with an octagonal PTFE coated magnetic stir bar was charged with 500 mg of HMF A (3.97 mmol), 640 μL of pyridine (7.92 mmol), 756 mg of p-toluenesulfonyl chloride (tosyl chloride, 3.97 mmol) and 10 mL of anhydrous methylene chloride. The reaction was stirred for 2 hours at room temperature. After this time, a light yellow solution was distinguished. The solution was poured into a 250 mL separation funnel, and diluted with 20 mL of methylene chloride and washed with 20 mL of 1N HCl. After vigorous agitation, the lower organic layer was discharged, residual aqueous phase extracted with 15 mL of methylene chloride, organic layers combined, dried with anhydrous magnesium sulfate and concentrated under reduce pressure, affording a 1.01 g of light yellow, crystalline solid (91% of theoretical). Thin layer, silica gel chromatography of the product mixture (100% ethyl acetate) displayed a single spot after development, Rf=0.62. ¹H NMR (CDCl₃, 400 MHz) δ (ppm) 9.64 (s 1H), 7.92 (d, J=7.2 Hz, 2H), 7.42 (d, J=7.6 Hz, 2H), 7.21 (d, J=5.6 Hz, 1H), 6.58 (d, J=6.4 Hz), 1H), 4.62 (s, 2H), 2.49 (s, 3H); ¹³C NMR (CDCl₃, 125 MHz) δ (ppm) 178.02, 156.35, 153.35, 146.99, 141.92, 130.43, 127.27, 112.14, 112.10, 64.85, 22.04. Collectively, the aforementioned data provides good evidence for the presence of HMF tosylate B as the primary product of the reaction.

### Example 3

### Synthesis of (5-formylfuran-2-yl)methyl methanesulfonate, HMF Mesylate B

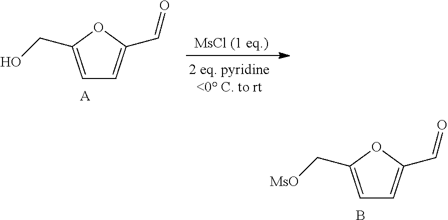

Experimental: An oven dried, 25 mL round bottomed flask equipped with an octagonal PTFE coated magnetic stir bar was charged with 500 mg of HMF A (3.97 mmol), 640 μL of pyridine (7.92 mmol), 307 μL of methanesulfonyl chloride (mesyl chloride, 3.97 mmol) and 10 mL of anhydrous methylene chloride. The reaction was stirred for 1.5 hours at room temperature, when the reaction was deemed complete via TLC (1:1 hexane/ethyl acetate, UV-Vis, absence of HMF band). The solution was then poured into a 250 mL separation funnel, and diluted with 20 mL of methylene chloride and washed with 20 mL of 1N HCl. After vigorous agitation, the lower organic layer was discharged, residual aqueous phase extracted with 15 mL of methylene chloride, organic layers combined, dried with anhydrous magnesium sulfite and concentrated under reduce pressure, affording 801 mg of a colorless, crystalline solid (98% of theoretical). Thin layer, silica gel chromatography of the product mixture (100% ethyl acetate) displayed a single spot after development, Rf=0.59. ¹H NMR (CDCl₃, 400 MHz) δ (ppm) 9.67 (s 1H), 7.16 (d, J=5.8 Hz, 1H), 6.66 (d, J=6.0 Hz), 1H), 4.60 (s, 2H), 3.10 (s, 3H); ¹³C NMR (CDCl₃, 125 MHz) δ (ppm) 178.06, 156.21, 153.54, 119.33, 112.04, 62.20, 39.42.

### B. HMF-Sulfonate Derivatives

### Example 1

### Amino-acid Based Amphiphile, Schiff-Base Modification (Plausible Surfactant)

### Synthesis of 2-(((5-((decyloxy)methyl)furan-2-yl)methyl)ammonio)acetate, 4

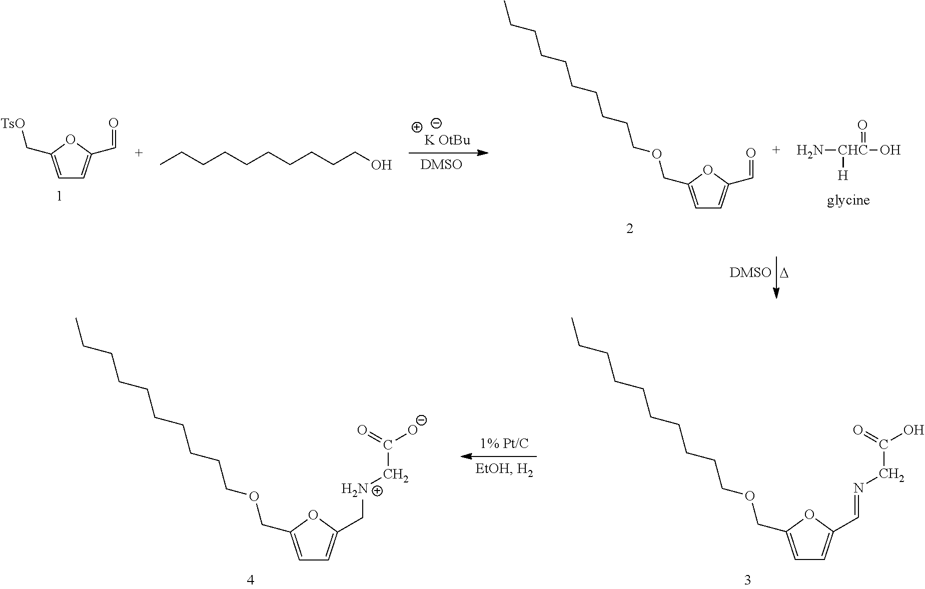

Experimental: A 25 mL, single-necked round bottomed flask equipped with an octagonal PTFE coated magnetic stir bar was charged with 250 mg of HMF-tosylate 1 (0.968 mmol), 161 mg of 1-decanol (1.01 mmol), 141 mg of potassium t-butoxide (1.25 mmol), and 10 mL of anhydrous DMSO. The reaction mixture was heated to 60° C. overnight. After this time, the mixture was allowed to cool to room temperature, an aliquot was removed, spotted on a silica gel TLC plate and developed with 1:1 ethyl:acetate hexanes as the eluent. One spot was manifest after UV illumination, Rf₁=0.72 adducing full conversion of 1 to the target ether 2. The solids were then filtered and surplus DMSO evaporated under vacuum, engendering 250 mg of clear semi-solid (96% of theoretical). This material (0.938 mmol) was charged to a dry 25 mL single necked round bottomed flask, along with 71 mg of glycine (0.940 mmol), and 5 mL of anhydrous DMSO. The flask was outfitted with a Liebig condenser capped with a 24/40 inner joint ground glass adapter, affixed to argon line. While stirring and under argon, the mixture is heated to 60° C. overnight. After this time, a 500 μL aliquot is removed, diluted with 1 mL of d⁶-DMSO and analyzed by ¹H NMR (400 MHz); the spectrum manifested a salient signal at 8.2 ppm in lieu of the signature aldehyde resonance frequency of 2 at 9.4 ppm, thus signifying that 2 had entirely converted to the targeted imine (Schiff-base) 3. Excess DMSO was then removed in vacuo, affording 299 mg (0.924 mmol) of a light yellow semi-solid (98% of theoretical). This material was then transferred to a 10 mL round bottomed flask, charged with 30 mg of 1% Pt/C and 10 mL of anhydrous ethanol and stoppered with a rubber septum. A thick-walled balloon was filled with H₂ and introduced via a 14″ needle transfixing the septum to the flask head space. Three full balloon volumes were charged to the headspace, through use of an outlet needle transfixing the septum to ensure all air had been evacuated. The mixture was stirred overnight with a full balloon of H₂. The next morning revealed the balloon to be nearly empty and a profusion of solid material observed at the bottom of the flask. Solids were filtered, dried, furnishing 275 mg (91%). A 5 mg sample was dissolved in D₂O and analyzed by ¹H NMR (400 MHz). The clean spectrum provided cogent evidence for the 2-(((5-((decyloxy)methyl)furan-2-yl)methyl)ammonio)acetate 4 from the following signals (δ, ppm): 7.84 (m, 2H), 7.64 (m, 2H), 7.22 (m, 2H), 6.37 (d, J=8.2 Hz, 1H), 6.25 (d, J=8.0 Hz, 1H), 4.92 (s, 2H), 4.85 (m, 2H), 4.47 (m, 2H), 3.19 (s, 3H), 2.22 (m, 2H), 1.71-1-65 (m, 16H), 0.82 (m, 3H).

### Example 2

### Amino-Acid Based Amphiphile, Thiol Substitution (Plausible Pre-Surfactant)

### Synthesis of 1-carboxy-2-(((5-formylfuran-2-yl)methyl)thio)ethanaminium 2,2,2-trifluoroacetate 3

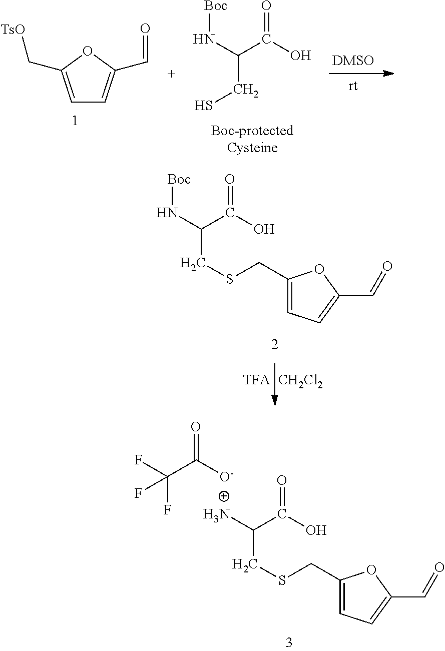 [SMILES: Cc1ccc(S(=O)(=O)OCc2ccc(C=O)o2)cc1 ; CC(C)(C)OC(=O)NC(CS)C(=O)O ; CC(C)(C)OC(=O)NC(CSCc1ccc(C=O)o1)C(=O)O ; "CH2Cl2" ; O=C([O-])C(F)(F)F ; [NH3+]C(CSCc1ccc(C=O)o1)C(=O)O]

Experimental: A single-neck, 25 mL round bottomed flask was charged with 222 mg of HMF-triflate 1 (0.860 mmol), 190 mg of Boc-cysteine (0.860 mmol), and 10 mL of anhydrous DMSO. While stirring, the mixture was heated to 50° C. overnight. After this time, an aliquot was removed, spotted on a TLC plate that was developed with 100% ethyl acetate as the eluent. One band was observed by UV illumination, located at the baseline (R_{f}=0) adducing full conversion of 1 to the Boc-cysteine derivative 2. Excess DMSO was then removed in vacuo, affording a white solid that, after drying under high vacuum for 3 days, weighed 261 mg (0.792 mmol, 92% of theoretical). This solid 2 was then charged to a 10 mL round bottomed flask equipped with a tapered magnetic stir bar followed by 5 mL of trifluoroacetic acid and 5 mL of anhydrous methylene chloride. After stirring for 30 min, excess trifluoroacetic acid and methylene chloride were removed under vacuum, bestowing a 265 mg of 3 as a light yellow solid (97% of theoretical). ¹H NMR analysis (D₂O, 400 MHz) revealed a very clean spectrum, exhibiting the following signals: δ (ppm) 9.68 (s, 1H), 7.11 (d, J=9.2 Hz, 1H), 6.97 (d, J=9.0 Hz, 1H), 4.01 (t, J=7.4 Hz, 1H), 3.67 (s, 2H), 2.11 (m, 2H). Collectively, these are cogent proof for derivative 3.

### Example 3

### Fatty-Acid Esters of HMF-Mesylate, Pre-Amphiphile

### Synthesis of (5-formylfuran-2-yl)methyl Oleate 2

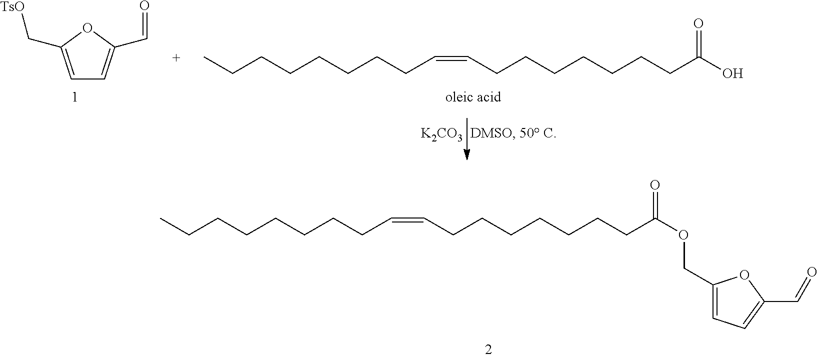 [SMILES: Cc1ccc(S(=O)(=O)OCc2ccc(C=O)o2)cc1 ; CCCCCCCC/C=C\CCCCCCCC(=O)O ; "K2CO3" ; CCCCCCCC/C=C\CCCCCCCC(=O)OCc1ccc(C=O)o1]

Experimental: A single-neck, 50 mL round bottomed flask equipped with a magnetic stir bar was charged with 300 mg of HMF-mesylate 1 (1.47 mmol), 415 mg of oleic acid (1.47 mmol), 610 mg of potassium carbonate (4.41 mmol) and 25 mL of anhydrous DMSO. The mixture was heated to 50° C. overnight. After this time, the solids were filtered and surplus DMSO evaporated under vacuum, producing a 561 mg of a light yellow semi-solid, that was deemed pure 2 by ¹H NMR analysis (400 MHz, CDCl₃): δ (ppm) 9.47 (s, 1H), 7.30 (d, J=9.4 Hz, 1H), 6.86 (d, J=9.0 Hz, 1H), 5.61 (m, 2H), 5.03 (s, 2H), 2.45 (t, J=7.2 Hz, 2H), 2.22 (m, 4H), 1.72 (m, 2H), 1.46-1.41 (m, 20H), 0.91 (m, 3H). Furthermore, a silica gel TLC plate was spotted with the product mixture, developed with 1:1 hexanes/ethyl acetate, and manifest a single band by UV illumination, Rf=0.72; the characteristic hands for both oleic acid (cerium molybdate stain, Rf=0.61) and HMF mesylate (UV, Rf=0.55) were patently absent.

### Example 4

### Alkylation of HMF Triflate via a Grignard Substitution, Synthesis of 5-pentylfuran-2-carbaldehyde 2

### Synthesis of 5-pentylfuran-2-carbaldehyde 2

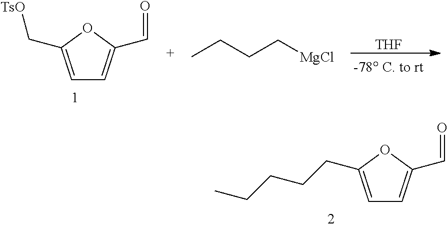

Experimental: A dry, single-neck 25 mL round bottomed flask equipped with a tapered PTFE coated magnetic stir bar was charged with 350 mg of HMF-triflate (1.36 mmol) and 10 mL of anhydrous THF. The flask was then capped with a rubber septum and immersed in a dry ice saturated acetone bath (˜−78° C.). While stirring, 678 μL of a 2M butyl magnesium chloride stock solution in diethyl ether (1.36 mmol) was added drop-wise over 5 minutes. The dry ice/acetone bath was then withdrawn and the reaction continued for 2 additional hours. After this time, an aliquot was extracted, spotted on a silica gel TLC plate and developed with a 1:1 ethyl acetate/hexanes eluent, disclosing a single spot with an Rf=0.70. The band specific to HMF-triflate, with an Rf=0.47, was noticeably absent. The solution was then concentrated under high vacuum for a period of 3 days, furnishing 221 mg of a clear, loose oil (98% of theoretical). ¹H NMR (400 MHz, CDCl₃) analysis revealed the following signals: δ (ppm) 9.32 (s, 1H), 7.27 (d, J=8.8 Hz, 1H), 6.92 (d, J=9.0 Hz, 1H), 2.64 (t, J=7.8 Hz, 2H), 1.81 (m, 2H), 1.31-1.29 (m, 4H), 0.82 (s, 3H).

### Example 5

### Synthesis of 5-(((ethylsulfonyl)oxy)methyl)furan-2-carboxylic Acid 2 via a Heyns Oxidation Protocol

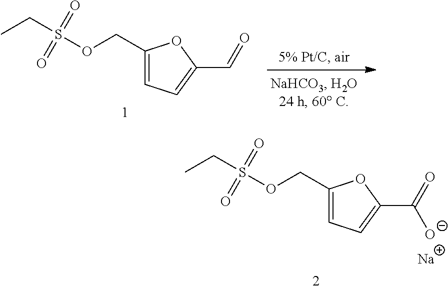

Experimental: Experimental: A single neck, 100 mL round bottomed flask equipped with a magnetic stir bar was charged with 1.00 g of HMF-Ethysulfonate (4.58 mmol) 1, 912 mg of 5% Pt/C (200 g/mol HMF), 2.31 g of NaHCO₃ (27.48 mmol) and 60 mL of deionized water. The neck of the flask was then capped with a rubber septum and an air inlet affixed via an 18 gauge stainless needle whose beveled tip was positioned near the bottom of the heterogeneous solution. In addition, six 2 inch, 16 gauge needles pierced the septum, utilized as air vents. While stirring, the flask was immersed in an oil bath and heated at 60° C. with vigorous sparging of air for a 24 hour time period. After this time, the Pt/C was removed by filtration and the aqueous residue analyzed by silica gel thin layer chromatography using 100% ethyl acetate developing solution and UV light for spot illumination. A single band, positioned at the baseline, was observed while that for HMF-ethylsulfonate (0.69 with an authentic sample) was absent, suggesting that 1 had been fully converted to the mono-sodium salt of FDCA 2. Cogent proof for the conversion of 1 arose from a) A clean ¹H NMR (400 MHz, D₂O) spectrum, where the signature aldehyde resonance signal at 9.33 ppm was absent and b) a clean ¹³C NMR (D₂O, 125 MHz) spectrum that manifested signals at 164.88, 155.33, 151.41, 120.18, 113.92, 64.02, 48.57, 13.63 ppm.

The present invention has been described in general and in detail by way of examples. Persons of skill in the art understand that the invention is not limited necessarily to the embodiments specifically disclosed, but that modifications and variations may be made without departing from the scope of the invention as defined by the following claims or their equivalents, including other equivalent components presently known, or to be developed, which may be used within the scope of the present invention. Therefore, unless changes otherwise depart from the scope of the invention, the changes should be construed as being included herein.

We claim:

1. A method of preparing a 5-(hydroxymethyl)furan-2-carbaldehyde (HMF) sulfonate, comprising: reacting a mixture of a 5-(hydroxymethyl)furfural (HMF) with a sulfonate species, and a reagent of either 1) a nucleophilic base or 2) a combination of a non-nucleophilic base and a nucleophile.

2. The method according to claim 1, wherein said sulfonate species is at least one of: a) trifluoromethanesulfonate anhydride (triflate), b) p-toluene-sulfonyl halide (tosylate), c) methane-sulfonyl halide (mesylate), d) ethanesulfonyl halide (esylate), and e) benzenesulfonyl halide (besylate).

3. The method according to claim 1, wherein wherein said nucleophilic base is at least one of: pyridine, dimethyl-aminopyridine, imidazole, pyrrolidine, and morpholine.

4. The process according to claim 1, wherein said non-nucleophilic base is an amine selected from the group consisting of: triethylamine, Hünig's base (N,N-diisopropylethylamine), N-methylpyrrolidine, 4-methylmorpholine, and 1,4-diazabicyclo-(2.2.2)-octane (DABCO).

5. The process according to claim 1, wherein said nucleophile is an alcohol (R—OH) moiety or 4-dimethylaminopyridine (DMAP).

6. The process according to claim 1, wherein when said reagent is a nucleophilic base, said reaction is conducted at an initial temperature of about 1° C. or less.

7. The process according to claim 6, wherein said initial temperature is in a range between about −15° C. and about −80° C.

8. The process according to claim 6, wherein when said sulfonate species is a trifluoromethanesulfonate anhydride, said initial temperature is at −10° C. or below prior to an addition of said HMF.

9. The process according to claim 1, wherein when said reagent is a combination of a non-nucleophilic base and a nucleophile, said reaction is conducted at about ambient room temperature or greater.

10. The process according to claim 1, wherein said process produces primarily 5-(hydroxymethyl)furan-2-carbaldehyde (HMF) sulfonate in molar yields of at least 50% from said HMF starting material.

11. A chemical compound comprising a 5-(hydroxymethyl)furan-2-carbaldehyde (HMF)-sulfonate having at least one of the following structures:

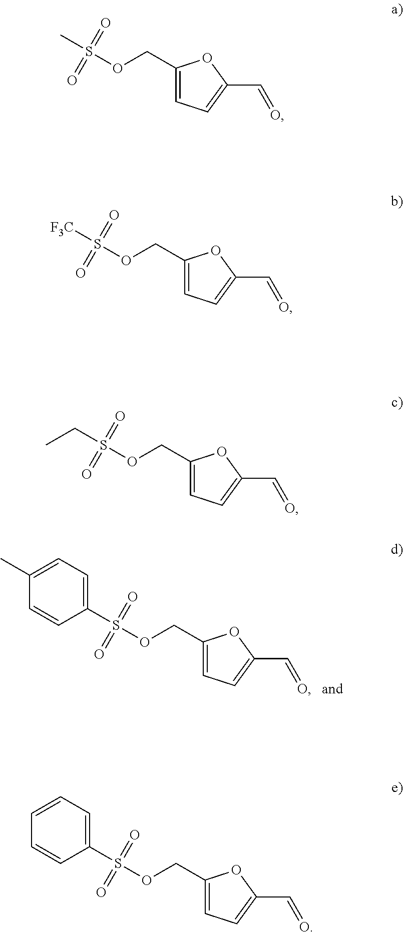 [SMILES: CS(=O)(=O)OCc1ccc(C=O)o1 ; O=Cc1ccc(COS(=O)(=O)C(F)(F)F)o1 ; CCS(=O)(=O)OCc1ccc(C=O)o1 ; Cc1ccc(S(=O)(=O)OCc2ccc(C=O)o2)cc1 ; O=Cc1ccc(COS(=O)(=O)c2ccccc2)o1]

12. A method of preparing a furanic derivative compound of a HMF-sulfonate comprising: reacting a mixture of a 5-(hydroxymethyl)furfural (HMF) with a sulfonate species and a reagent of either 1) a nucleophilic base or 2) a combination of a non-nucleophilic base and a nucleophile with heat to synthesize a 5-(hydroxymethyl)furan-2-carbaldehyde (HMF)-sulfonate; and transforming said 5-(hydroxymethyl)furan-2-carbaldehyde (HMF)-sulfonate into a furanic derivative compound.

13. The method according to claim 12, wherein said sulfonate species is at least one of a) trifluoromethanesulfonate (triflate), b) p-toluene-sulfonate (tosylate), c) methane-sulfonate (mesylate), d) ethanesulfonate (esylate) and e) benzenesulfonate (besylate).

14. The method according to claim 12, wherein said transforming to said furanic derivative compound comprises: reacting said HMF-sulfonate with least one of the following: a) an alkyl- or aryl-magnesium halide (Grignard reagents) or organolithium compound (lithiates), b) an organic acid, c) an alkyl halogen, d) an oxidizing reagent, e) a reducing reagent, f) a Schiff-base, g) a thiol and h) a Wittig reagent to perform, respectively, at least one of the following: an alkylation, esterification, halogenation, oxidation, reduction, Schiff-base modification, thiolation, and Wittig olefination.

15. (canceled)

16. (canceled)

17. (canceled)

18. (canceled)

19. (canceled)

20. (canceled)

21. (canceled)

22. (canceled)

23. A furanic derivative compound, made according to the method of claim 12, is at least one of the following:

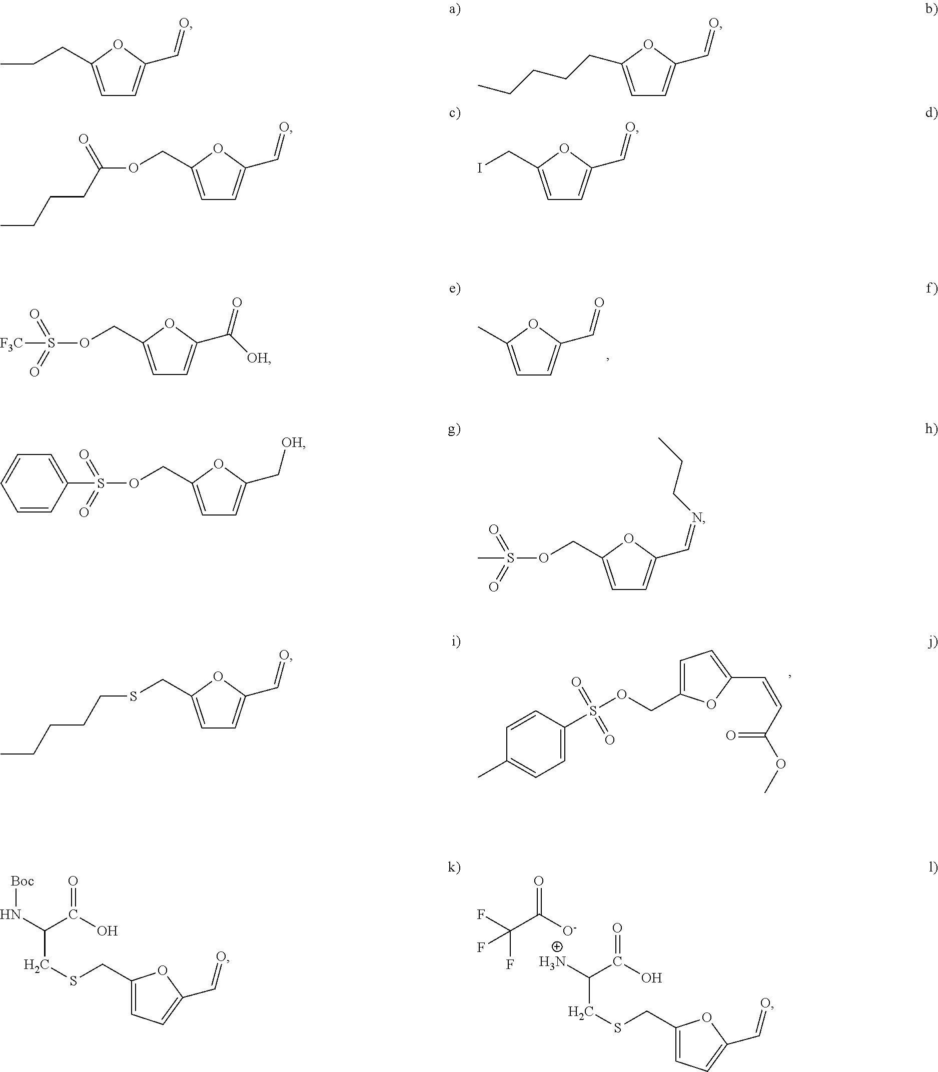

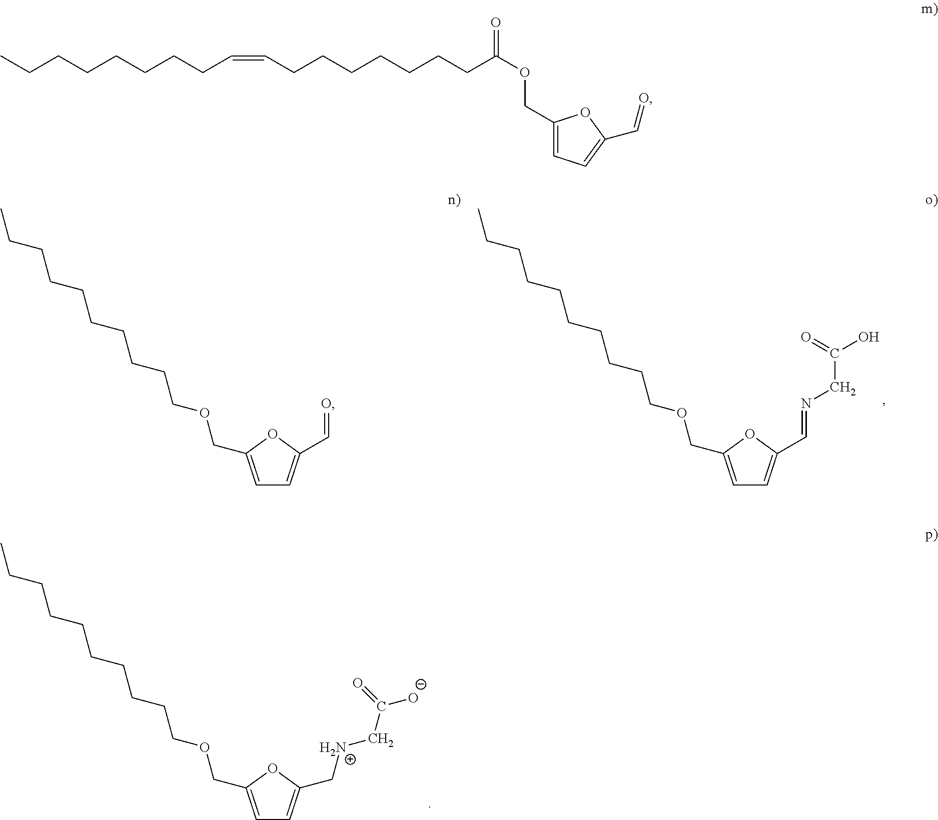 [SMILES: CCCCCCCC/C=C\CCCCCCCC(=O)OCc1ccc(C=O)o1 ; CCCCCCCCCCOCc1ccc(C=O)o1 ; CCCCCCCCCCOCc1ccc(/C=N/CC(=O)O)o1 ; "," ; CCCCCCCCCCOCc1ccc(C[NH2+]CC(=O)[O-])o1]

24. A furanic derivative compound, transformed according to claim 14 by at least:
a) alkylation, having a general formula: 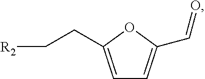
where R₂ is an alkyl, allyl or aryl species;
b) esterification, having a general formula: 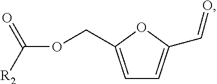
where R₂ is an alkyl, allyl or aryl species;
c) halogenation, having a general formula: 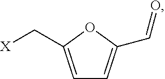
where X is a halogen;
d) oxidation, having a general formula: 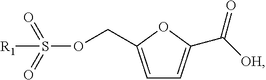
where R₁ is a —CH₃, —CF₃, —C₆H₄CH₃, —C₂H₅, or —C₆H₅;
e) reduction, having a general formula: 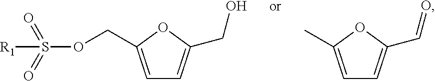
where R₁ is a —CH₃, —CF₃, —C₆H₄CH₃, —C₂H₅, or —C₆H₅;
f) Schiff-base modification, having a general formula: 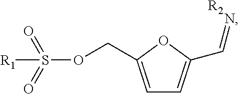
where R₁ is a —CH₃, —CF₃, —C₆H₄CH₃, —C₂H₅, or —C₆H₅, and R₂ is an alkyl, allyl or aryl species;
g) thiolation, having a general formula: 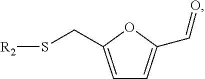
where R₂ is an alkyl, allyl or aryl species;
h) Wittig olefination, having a general formula: 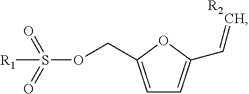
where R₁ is a —CH₃, —CF₃, —C₆H₄CH₃, —C₂H₅, or —C₆H₅, and R₂ is an alkyl, allyl or aryl species.
